(19) United States
(12) Patent Application Publication
(10) Pub. No.: US 2017/0016884 A1
(43) Pub. Date: Jan. 19, 2017

(54) A NOVEL, HIGH-THROUGHPUT, NANOTOPOGRAPHIC PLATFORM FOR SCREENING CELL MIGRATORY BEHAVIOR

(71) Applicant: THE JOHNS HOPKINS UNIVERSITY, Baltimore, MD (US)

(72) Inventors: Christopher Smith, Baltimore, MD (US); Andre Levchenko, Madison, CT (US); Deok-Ho Kim, Seattle, WA (US); Alfredo Quinones-Hinojosa, Bel Air, MD (US)

(21) Appl. No.: 15/125,041

(22) PCT Filed: Mar. 10, 2015
(86) PCT No.: PCT/US2015/019692

§ 371 Date: Sep. 9, 2016

Related U.S. Application Data
(60) Provisional application No. 61/950,446, filed on Mar. 10, 2014.

(51) Int. Cl. G01N 33/50; B01L 3/00; G01N 33/574; G01N 33/483

(52) U.S. Cl. CPC G01N 33/5029; G01N 33/4833; B01L 3/5085; G01N 33/57407; G01N 33/574; B01L 2300/0829; B01L 2300/0896; B01L 2300/163; B01L 2300/0822

(57) ABSTRACT

The present invention is directed to creating a highly versatile, high throughput, and convenient platform for interrogating migration phenotypes of GB cells in the context of diverse environmental parameters. Specifically, engineered substrates are employed to mimic the mechanical signals of natural ECM topography, and combine these tools with chemical cues presented in soluble and immobilized forms (PDGF and laminin, respectively). This platform achieves far greater resolution and sensitivity in migration analyses than do commonly used methods. More importantly, it can provide highly informative, patient specific results regarding tumor progression in vivo. Furthermore these capabilities strongly convey the immense clinical, prognostic potential of this simple test.

### CROSS REFERENCE TO RELATED APPLICATIONS

This application claims the benefit of U.S. Provisional Patent Application No. 61/950,446 filed Mar. 10, 2014, which is incorporated by reference herein, in its entirety.

### GOVERNMENT SUPPORT

This invention was made with government support under R01NS070024 awarded by the National Institutes of Health. The government has certain rights in the invention.

### FIELD OF THE INVENTION

The present invention relates generally to laboratory testing. More particularly, the present invention relates to a device and method for screening for cell migratory behavior.

### BACKGROUND OF THE INVENTION

Personalized medicine can benefit from patient-specific analysis of cell and tissue properties, especially if such tests are of prognostic value. Aggressive cancers, such as glioblastoma (GB), are of particular interest due to the heterogeneous nature of individual tumors and the high incidence of recurrence following surgical resection. Genomic and proteomic profiling can provide a wealth of information about tumor samples, including cancer-causing abnormalities and clinically relevant subclasses. However, this information may not be easily interpretable or predictive of certain complex phenotypes, such as invasive growth and enhanced migratory cell dissemination. In spite of recent progress, high throughput genomic and proteomic analysis of multiple samples with single cell resolution is still not within reach of clinical applications. Thus, significant value may be offered by a complementary approach: the analysis of phenotypic properties of tumor cells. If properly designed, such an analysis can be rapid, simple, high throughput, and easily conducted on the scale of individual cells. Its utility will depend on the prognostic value attained, and on how predictive a certain phenotype is within the context of a specific tumor.

Aggressive cell migration and dispersal is common to GB, helping the disease overcome standard treatments, including surgery, radio-, and chemotherapies. As with many other cancers, individual GB cells can spread from the primary tumor bulk, avoid detection, and reconstitute tumor masses in different areas of the body. These migratory and invasive capacities are governed by a number of genetic and environmental variables. Growth factors, e.g. platelet-derived growth factor (PDGF), have emerged as potential enhancers of migratory potential in GB, although this function is not clearly decoupled from their effects on tumor cell proliferation. Various features of the extracellular matrix (ECM) have also been implicated in modulation of cell migration. ECM proteins, such as laminin, are known to regulate cell motility, specifically in neural tissues. In addition, migrating cells can be guided by a variety of mechanical cues presented by the ECM. These stimuli emerge through tissue structures ranging, in size, from nanometers (nm) to microns (ii m). Together, these findings suggest that phenotypic analysis of the tumor samples must recapitulate the complexity of the chemical and mechanical features present in the tumor micro- and nano-environment.

Accordingly, there is a need in the art an experimental platform that would more realistically model the mechano-chemical cellular milieu, yet remain simple and accessible to allow practical, high throughput use.

### SUMMARY OF THE INVENTION

The foregoing needs are met, to a great extent, by the present invention which provides a device for cell migration including a multi-well plate having a number of well chambers. The well chambers include at least one wall defining the well chamber. The device also includes a nanopatterned surface. The nanopatterned surface is positioned on the at least one wall defining the well chamber. The nanopatterened surface is configured to mimic the topographic structure of a natural cell environment.

In accordance with an aspect of the present invention, the nanopatterned surface further comprises parallel ridges. The parallel ridges are approximately 350 nm wide and approximately 500 nm high and spaced apart 1.5 nm. The nanopatterned surface is formed directly into the at least one wall defining the well chamber. The nanopatterened surface is formed onto a glass coverslip configured to be coupled to the multi-well plate.

In accordance with an aspect of the present invention, the nanopatterned surface is configured to mimic the mechanical signals of natural ECM topography. The nanopatterned surface configured to mimic the mechanical signals of natural ECM topography is combined with chemical cues. The chemical cues can be soluble and immobilized. The device includes a coating of laminin, and the concentration of laminin in the coating can be varied. The device can also include a coating of platelet derived growth factor (PDGF), and the concentration of PDGF in the coating can also be varied. The device can be configured for cell migration to test migration of glioblastoma. The device is configured to provide patient-specific results regarding tumor progression. The device is also configured for diagnosis of brain tumors.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A-1C illustrate schematic diagrams of an exemplary embodiment of the multi-well plate, according to an embodiment of the present invention.

FIG. 1D illustrates imaging of an exemplary cell plate according to an embodiment of the present invention.

FIGS. 1E-1G illustrate graphical views of cell migration, according to an embodiment of the present invention.

FIGS. 2A-2D illustrate graphical views of the quantification of migration measured by speed, alignment and persistence of GB318 cells cultured on the nano patterned surface 2B with varying laminin coating concentrations and PDGF coating concentrations, according to an embodiment of the present invention.

FIGS. 3A-3F illustrate graphical views of nanopatterned platform hererogenieity, according to an embodiment of the present invention.

FIGS. 4A-4F illustrate graphical views of migratory response to PDGF to predict tumor characteristics in vitro and in vivo, according to an embodiment of the present invention.

FIGS. 5A-5F illustrate GB migratory response to PDGF to correlate with patient tumor characteristics, according to an embodiment of the present invention.

FIG. 6 illustrates a table of trends in patient tumor characteristics, according to an embodiment of the present invention.

FIGS. 7A-7E illustrate that a multi-well nanopatterned platform induces changes in cell morphology and migration, according to an embodiment of the present invention.

FIGS. 8A and 8B illustrate graphical views that nanopatterned platforms enable higher sensitivity to immobilized factors, according to an embodiment of the present invention.

FIGS. 9A and 9B illustrate graphical views that nanopatterned surfaces elicit dose response of GB to varying concentrations of PDGF, according to an embodiment of the present invention.

FIGS. 10A-10C illustrate graphical views that GB migratory response to PDGF predicts receptor expression, according to an embodiment of the present invention.

FIGS. 11A-11E illustrate that migratory response to PDGF corresponds to tumor formation in vivo, according to an embodiment of the present invention.

FIGS. 12A-12E illustrate that migratory response to PDGF correlates to proliferative response, according to an embodiment of the present invention.

FIGS. 13A-13C illustrate graphical views that migratory behavior informs patient tumor characteristics, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to creating a highly versatile, high throughput, and convenient platform for interrogating migration phenotypes of GB cells in the context of diverse environmental parameters. Specifically, engineered substrates are employed to mimic the mechanical signals of natural ECM topography, and combine these tools with chemical cues presented in soluble and immobilized forms (PDGF and laminin, respectively). This platform achieves far greater resolution and sensitivity in migration analyses than do commonly used methods. More importantly, it can provide highly informative, patient specific results regarding tumor progression in vivo. Furthermore these capabilities strongly convey the immense clinical, prognostic potential of this simple test.

Additionally, the present invention includes a multi-well platform that employs nanometer-scale patterned surfaces to model topographic features of extracellular matrix. Using glioblastoma as an experimental model, the multi-well platform is tested with respect to the migratory responses of cells to both soluble and immobilized stimulatory factors. In comparison to classical methods, our platform induces changes in cell morphology and migration that are more characteristic of in vivo phenomena. The device and method of the present invention also provides more sensitive and detailed information about migratory responses to immobilized laminin and soluble platelet-derived growth factor (PDGF). Furthermore, this critical information can be used to predict other phenotypic features of cells, e.g. protein expression, as well as and characteristics of patient tumors in vivo. Thus, our multi-well, nanopatterned platform enables rapid, simple screening of migratory behavior that can be employed for research and clinical applications, such as personalized medicine.

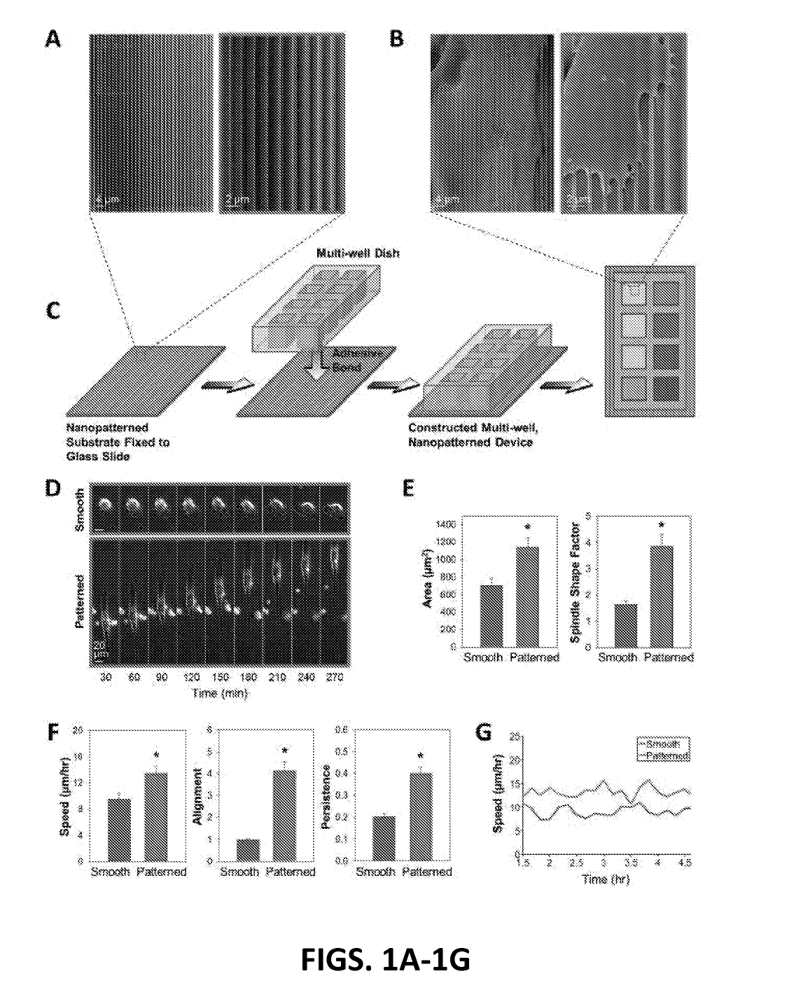

We have previously used UV-assisted capillary force lithography (CFL) to develop biologically relevant cell adhesion substrata, mimicking nanometer (nm) scale features of the ECM. Employing similar methods in this study, we fabricated topographic patterns comprising regular, parallel ridges 350 nm wide, 500 nm high, spaced 1.5 μm apart (FIGS. 1A and 1B). We designed this pattern to closely model aligned, elongated tissues found in ECM generally, and in the brain tissue more specifically. Invading glioma cells are known to preferentially migrate adjacent to such elongated fibers, as well as other anisotropic structures, e.g. axons and blood vessels. These ECM-rich features can range from 20 nm in diameter, e.g. collagen fibrils, to several microns across, e.g. myelinated axons. While we have examined cell migratory responses to various sizes and shapes of topographic cues, much of the data shown here employs a single substrate design of an intermediate size to investigate the combined effects of chemical and mechanical stimuli. This simplified approach was taken allow proof-of-concept analysis of the capabilities of this system.

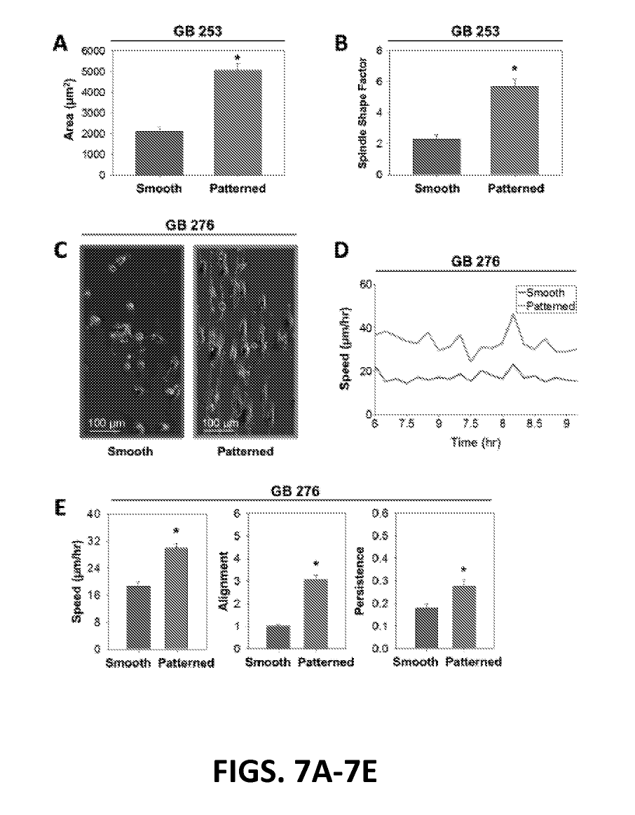

The pattern was constructed of poly(urethane acrylate) (PUA), and bonded to glass coverslips to allow optical imaging. To achieve high throughput screening of different conditions, these nanopattern coated coverslips were integrated with multi-well chambers, allowing simultaneous observation of multiple conditions (FIG. 1C). In most experiments, patterns were first coated with an ECM protein laminin to allow cell attachment (in a subset of experiments, the laminin concentration was varied). Dissociated primary human GB cells derived from patients (GB 253, GB 276, GB 318) were then plated at low density to allow time lapse observation of individual cell movements while minimizing cell collisions and collective interactions (FIG. 7C).

This design of the experimental platform had several important advantages vs. existing methods to score cell migration. First, the cell motility was observed to become essentially one-dimensional on the nanopatterned substrata (see detailed analysis below), allowing for an easier analysis of the cell migration propensity, while also mimicking the in vivo migration substrata. Second, the device construction permitted optical scoring of cell motility throughout the experiment, rather than detection of the initial and final distribution of cell density, as in many other assays (e.g., the trans-well migration assay). Third, the single-cell resolution allowed us to model migration following epithelial-to-mesenchymal transition (EMT), wherein cells detach and move individually. This critical step in cancer progression is overlooked by many standard assays that observe only collective dispersion. Finally, integration with multi-well format, accessible to common manual or automated liquid handling techniques, can make both condition screening and cell scoring a high throughput experiment.

Previous studies have highlighted the importance of cell polarity and morphology in migration, some specifically noting the elongated, spindle shape of invading glioma cells in vivo. Thus, in our initial tests, we compared the morphology of GB cells cultured on the nanoridge pattern to that of cells grown on a smooth PUA substrate. Mirroring observations of other cell types, GB 318 cells polarized and elongated parallel to the pattern, with processes enveloping the nanoridges (FIG. 1B). These changes resulted in significantly increased cell area and spindle shape factor in comparison to cells cultured on smooth substrata (FIG. 1E). These results suggest that our ridged substrate induces migratory behavior more characteristic of that seen in vivo.

Prior work has also demonstrated effects of nanotopography on cell migration (14, 20, 21). Therefore, we examined changes in GB cell motility induced by incubation on a nanoridge substratum. Consistent with a tendency of mobile cells to align parallel to oriented topographic structures, the direction of cell migration was strongly biased along the axis of the ridge pattern (FIG. 1D, FIG. 7C). Compared to movements on smooth substrata, migration on the nanoridge pattern was enhanced based on the three metrics scored: speed, alignment, and persistence (FIG. 1F). Alignment (measured here as the ratio of the distance moved parallel to the pattern vs. distance perpendicular to the pattern) describes how strongly cells interact with the underlying substrate. Both spindle shape factor and alignment have been shown to correlate with the structure and strength of cell-substrate adhesion complexes, which are critical regulators of cell motility and morphology. Persistence distinguishes random, exploratory motility from continuous motion in a particular direction, a critical migratory mode for tumor dispersal. This was quantified as the ratio of the shortest starting point-to-endpoint distance compared to the total distance traveled in the complete cell trajectory. GB cells on the smooth substratum repeatedly spread and contracted lamellipodia-like extensions, without making significant progress in any given direction; whereas cells on the nanoridge pattern often traversed several cell lengths along the ridge axis before changing directions (FIG. 1D, FIG. 7C). Our experiments yielded results for cell migration over 10 to 36 hours for a given experiment. Population averages of instantaneous speeds demonstrated that migratory enhancements were highly consistent for the duration of our experiments, and were not time-dependent (n>60 cells, FIG. 1G). We confirmed each of these findings for at least one additional primary human GB sample from intraoperative tissue (GB 253 and GB 276, FIGS. 7A-7E). These results suggested that our platform can indeed mediate enhanced cell polarization and migration in a manner that more closely mimics conditions in vivo and facilitates quantification.

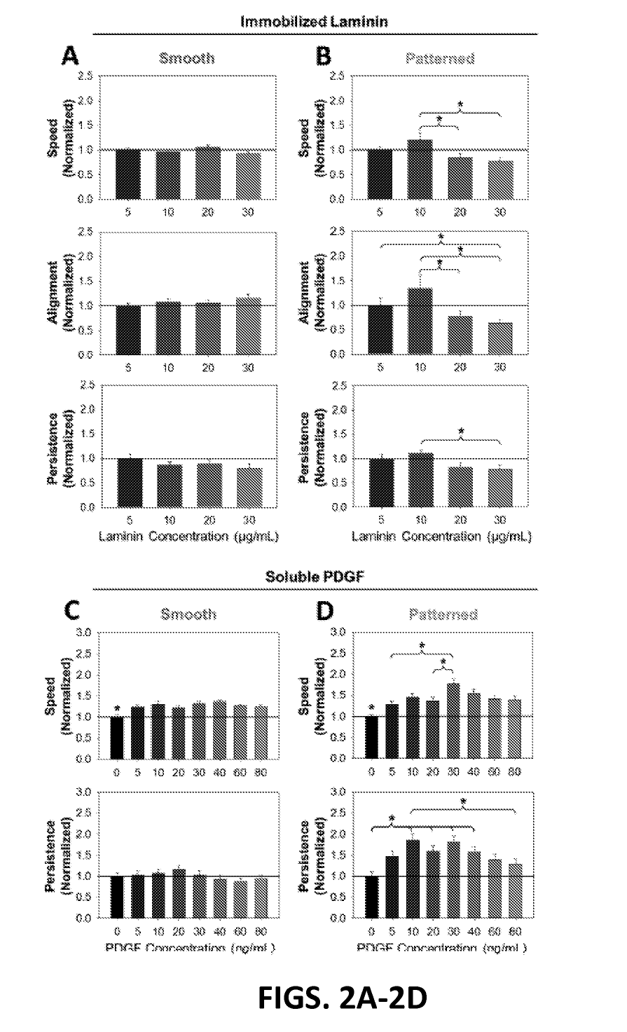

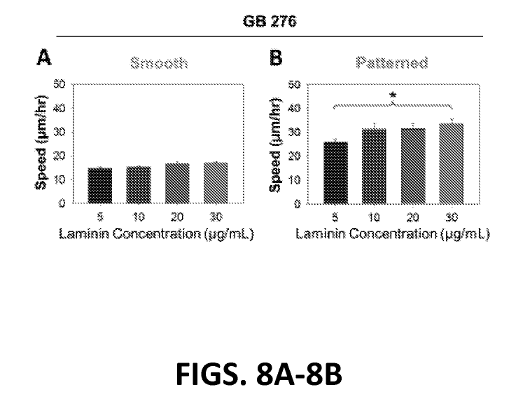

Having examined the effects of the nano-scale topography on polarity and migration of GB cells, we next explored how these parameters of cell morphology and motility can be modulated by other stimuli. In particular, we examined migratory responses of GB cells to numerous, simultaneously analyzed, variations of immobilized and soluble, chemical factors. We first focused on responses to the surfaceimmobilized, ECM protein laminin This critical regulator of neural cell migration is expressed in vascular niches of the brain, and has been implicated in glioma maintenance. Many studies have employed immobilized laminin for assaying cell growth and migration in vitro, in some cases finding enhanced responses of human neural cells vs. responses to other ECM proteins. Using the multi-well format, we examined the migration of GB cells on substrata coated with various concentrations of laminin in a single assay. We conducted this analysis using both nanopatterned and flat surfaces. When analyzed on nanopatterened substrata, results were consistent with previous findings examining the effect of the surface ECM density on cell migration. GB 318 cells displayed a biphasic response to the laminin concentration, with the optimal speed, alignment, and persistence at intermediate values (FIG. 2B). In contrast, on flat substrata, we did not observe statistically significant differences in migration at varying laminin densities (FIG. 2A). Similar results were obtained using GB sample 276 (FIG. 8A and FIG. 8B). In later experiments, we used a single laminin coating concentration (10 μg/mL) while varying other components of the cell environment.

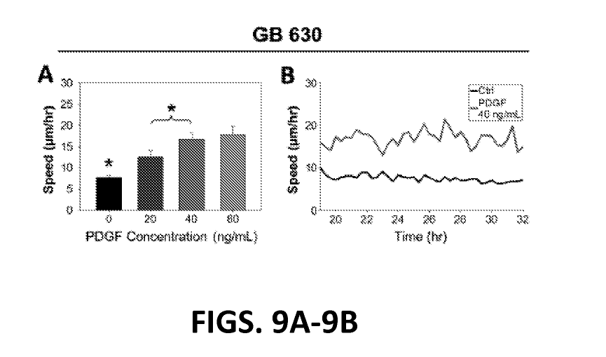

Next we analyzed migratory responses of GB cells to a soluble cue. Platelet-derived growth factor-AA (PDGF) signaling is involved in various aspects of glioma progression, and has been suggested to affect GB migration and invasion. In a single assay, we examined the effects of seven PDGF concentrations presented uniformly in serum-free culture medium. When cells were analyzed on nanopatterned system, there was a clear graded dose dependence of response in migration speed and persistence, with maximal (up to 2-fold higher than control) motility at intermediate concentrations (FIG. 2D and FIGS. 9A and 9B). In contrast, analogous experiments conducted on flat substrata produced much more limited responses to PDGF exposure (FIG. 2C), without strong sensitivity to PDGF concentration. Similar dependencies were seen for persistence of cell motility. Taken together, these results demonstrate that the described methodology enables more sensitive observation of cellular responses to soluble and immobilized factors than do standard migration analysis techniques.

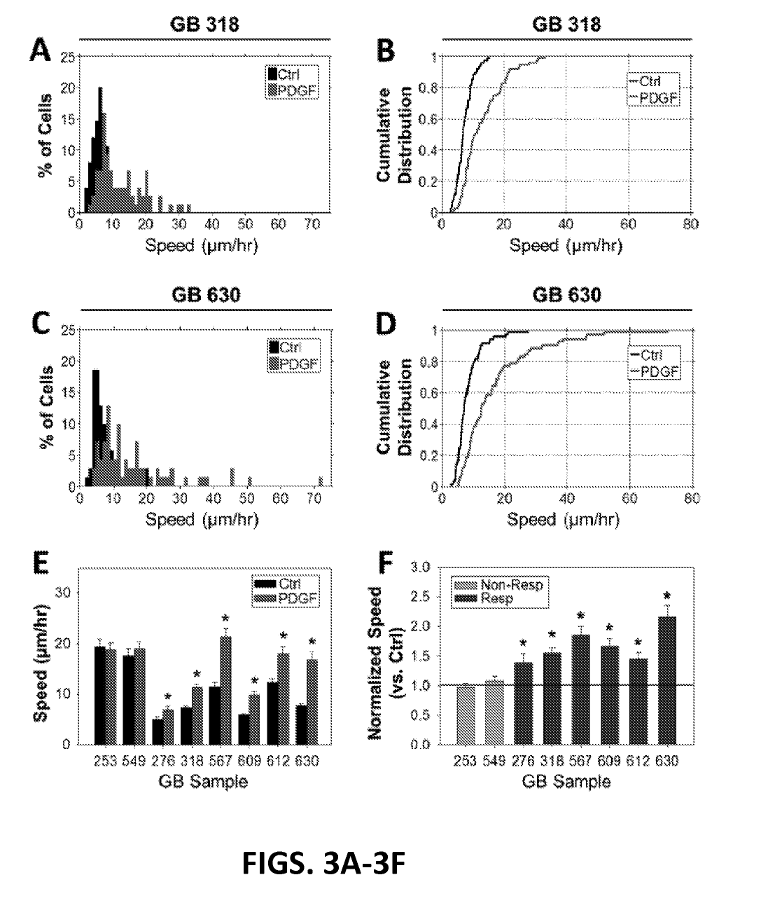

We next assayed the degree of cell-cell variability in migratory response. Such information is particularly critical in cancer biology, wherein distinct genotypic and phenotypic populations of cells can coexist within the same tumor. This type of heterogeneity can help select cell subpopulations promoting tumor growth, progression, and therapeutic resistance (1). GB in particular, is known to have populations with distinct expression profiles of receptor tyrosine kinases, particularly the PDGF Receptor-alpha (PDGFRα). We thus took advantage of the single cell resolution of the assay to quantify the distribution of cell speed in control vs. PDGF-exposed conditions, indeed finding that only a fraction of the cell population responded to PDGF (FIGS. 3A and 3C). Most cells maintained slower speeds (≦15 μm/hr) even in the presence of PDGF AA. This analysis was also informative about the differences in responses between GB samples derived from distinct patients. The histogram of GB 630 migration speeds had a broader distribution with a much longer tail extending into the region of higher cell speeds in comparison to the GB 318 histogram (FIGS. 3B and 3D). These measurements also demonstrated that a sub-population of cells within sample GB 630 can achieve higher speeds upon PDGF stimulation. At the same time, the bulk of distributions for both samples showed considerable overlap, with roughly 70% of GB 318 cells and 60% of GB 630 cells maintaining slower speeds (≦15 μm/hr). We note that single-cell information of such a high throughput and precise nature cannot be easily obtained using standard techniques such as trans-well chambers.

Subsequently, we further investigated difference in migratory behaviors among distinct tumors. The described multi-well, nanotopographic platform allows miniaturization of complex analyses, requiring only ˜103 cells for individual experiments. This is particularly beneficial for screening of primary or intraoperative human tissues, wherein cellular resources may be considerably limited. Taking advantage of this feature, we screened migratory responses to PDGF of numerous primary GB samples, obtained from different patients, simultaneously. We observed clear differences in the speed of cell motility across the spectrum of these 8 samples, both in the presence and absence of PDGF (FIG. 3E). Most

GB samples responded to intermediate concentrations of PDGF (40-50 ng/mL) with significantly increased speeds. To quantify responsiveness, we used the ratio of migration speed upon PDGF stimulation to the speed of unstimulated cells of the same sample (FIG. 3F). These measurements allowed separation of the samples into ‘responding’, i.e. displaying statistically significant increases in speed (of approximately 15-100%), and ‘non-responding’, which showed no significant enhancement. With these collective results, our methodology highlights critical differences in migratory behavior both within and across cell populations.

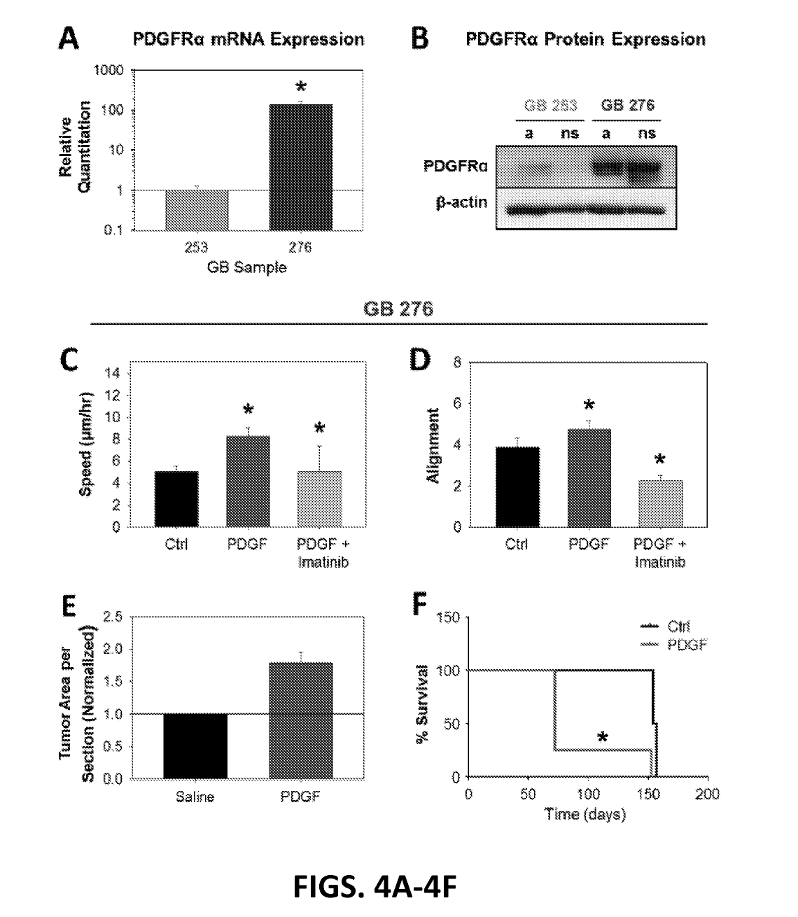

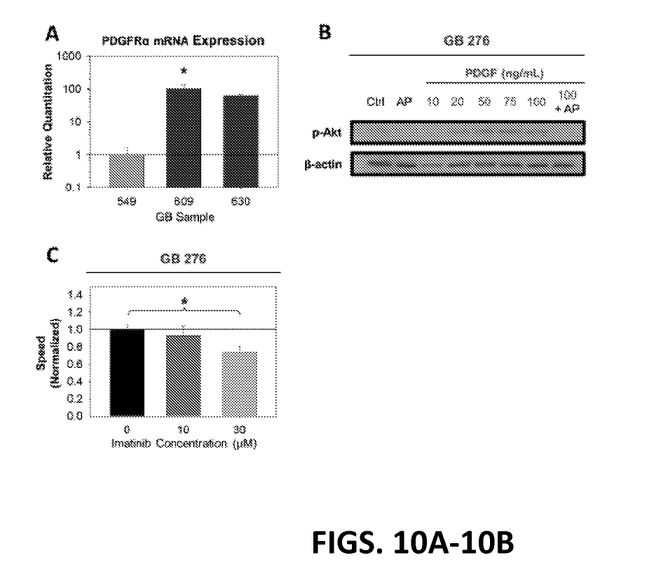

We next investigated whether the nanotopographic migration analysis could inform other crucial tumor characteristics, first examining cell phenotype. PDGFRα, whose amplification has been specifically observed in GB, is the exclusive receptor for the PDGF-AA isoform that we employed in this study. Thus we used RT-PCR analysis to determine if differential expression of PDGFRαcoincided with the disparate responses to PDGF in our GB samples. As predicted, we observed significantly higher expression in the ‘responding’ sample GB 276, compared to a ‘non-responding’ sample, GB 253 (FIG. 4A). We confirmed similar results in additional GB samples (GB 549, 609, and 630, FIG. 10A). Using immublotting, we found greater protein expression in GB 276, both when cells were cultured as adherent astrocytes (A) and floating neurospheres (NS) (FIG. 4B). To further ascertain that a receptor tyrosine kinase, such as PDGFRα, was functionally involved, we employed the small molecule inhibitor Imatinib. This inhibitor is known to interact with PDGFRα (2, 39), and has been shown to inhibit GB growth in vitro and in vivo (40). We examined its effects on the PDGF-stimulated migration of a ‘responding’ sample (GB 276), and observed attenuation of enhancements in speed and alignment (FIGS. 4C and 4D). We also assayed phosphorylation of the downstream effector Akt via immunoblotting, and observed PDGFdependent, Imatinib-sensitive activation (FIG. 10B). As these observations suggest, the differential GB migratory responses to PDGF can illustrate PDGFRαexpression and functional activity. The phenotypic response, while correlated with alteration in signaling output, is a more direct characteristic of the cell migratory response, accessible by quantitative analysis at the single cell level. FIG. 10C illustrates migration speed of GB276 cells cultured on a nanpatterned platform in the presence of Imatinib.

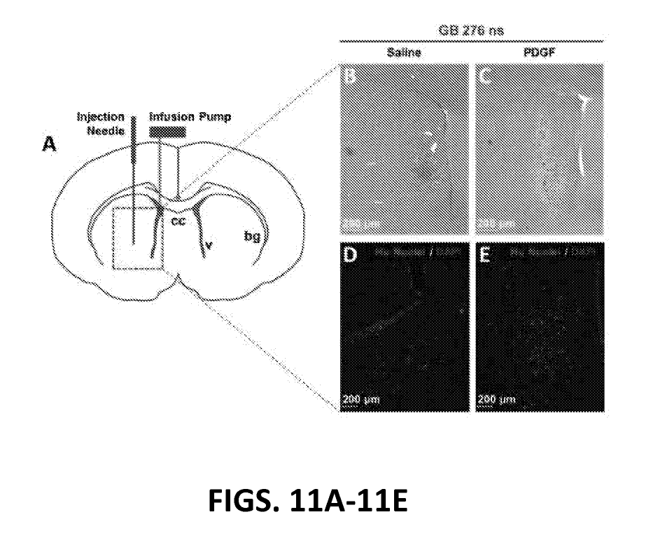

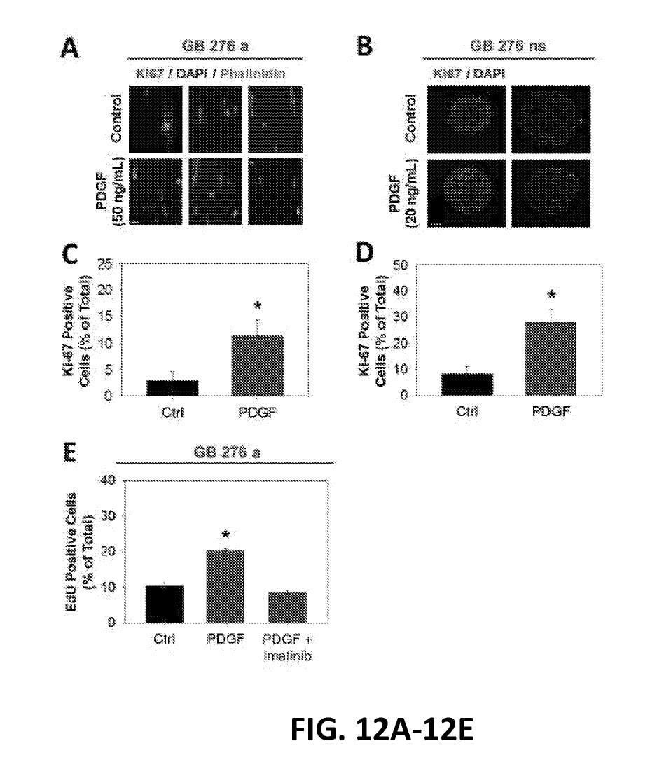

The PDGF-induced migratory response observed using the described nanotopographic platform also correlated with de novo tumor formation in vivo. We have previously described mouse tumor xenograft models involving intracranial injections of human GB cells as diagramed in FIG. 11A. We combined this approach with exogenous PDGF exposure to examine tumor formation and survival. Quantification of tumor size, and qualitative analysis by a blinded neuropathologist, suggested that continuous exposure of tumor xenografts to PDGF via infusion pump generated larger, more invasion tumors with more eccentric shapes (n=2, FIG. 4E, FIGS. 11B-11E). These features are indicative of migration along fiber tracts. In an alternative approach, GB 276 cells were cultured as neurospheres in the presence or absence of PDGF for three weeks prior to intracranial injections. We subsequently observed significantly reduced survival in mice injected with PDGF-preconditioned cells (n=4, FIG. 4F). While our in vitro migratory analysis of GB 276 suggested a more invasive phenotype upon PDGF exposure, we cannot rule out that responses other than migration contributed to the poorer prognosis in vivo. In vitro analysis of GB 276 cells showed that proliferation also correlated with the PDGF-induced migratory response (FIGS. 12A-12E). Our findings here are consistent with tumor induction and progression witnessed in animal models in previous investigation. Moreover, they demonstrate that our platform can provide valuable information, indicative of in vivo characteristics, through simple in vitro phenotypic analyses.

The degree of cell migration may reflect the propensity for invasive tumor spread.

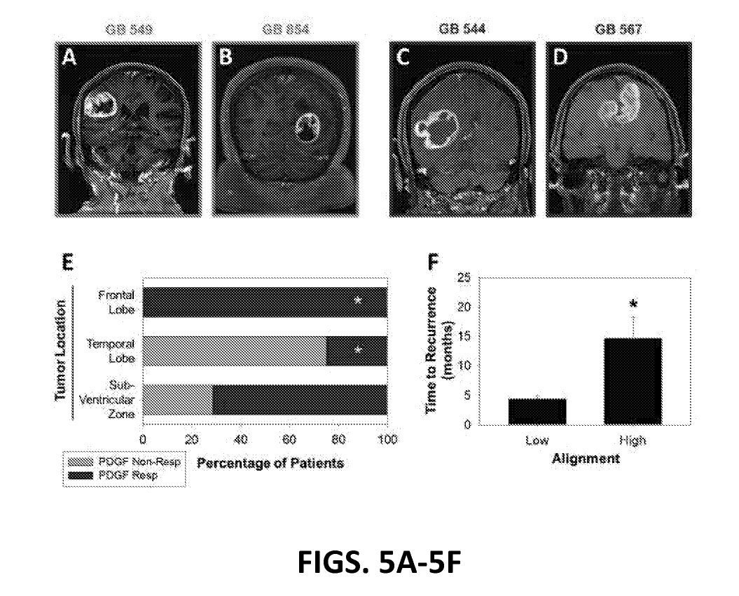

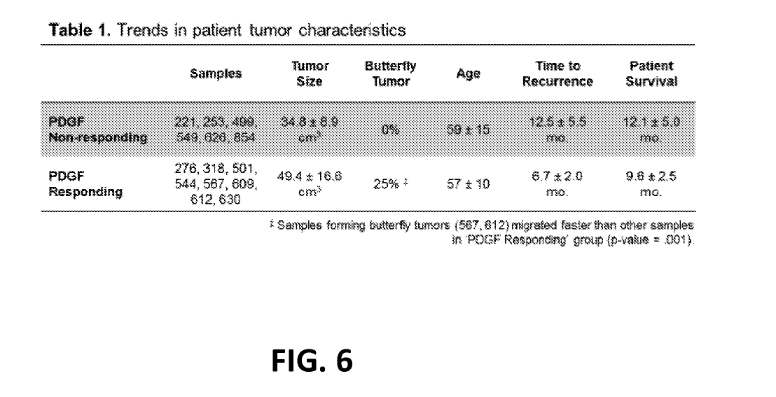

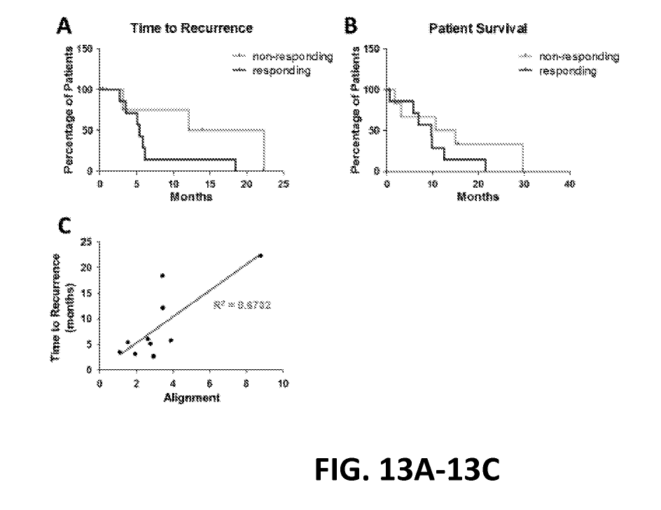

Since the samples analyzed in our tests were derived from patients whose clinical history was available for querying, we explored if the migratory responses observed in the described nanotopographic platform corresponded with clinical aspects of tumor progression. Strikingly, when contrasting different patient tumors from which cell samples were obtained, we found trends of more invasive and aggressive tumor features in GB samples that responded to PDGF, as judged by the cell migration analysis (FIG. 5, FIG. 6, FIGS. 13A-13C). This result further supported, and provided a mechanistic underpinning, to the prior observations that PDGF signaling can affect brain tumor grade and prognosis in patients, and can lead to reduced tumor latency and more invasive phenotypes in vivo. Some characteristic differences in tumor features were visualized in MRI imaging of the patients (FIG. 5A-D).

Tumors from the PDGF-‘responding’ group (FIG. 5C and 5D) appeared larger and more spread-out. Furthermore, formation of the so-called ‘butterfly’ tumors, wherein cells migrate across the corpus callosum to the opposite side of the brain, occurred only in the PDGF-‘responding’ group (FIG. 6, FIG. 5D note the butterfly tumor occurrence spanning both brain hemispheres). Interestingly, within this group, the two samples that formed butterfly tumors (GB 567, GB 612) were also the fastest moving on the surface of laminin (FIG. 6, FIG. 3E). We also observed statistically significant differences in the anatomical location of the tumors, with all frontal lobe-derived GB samples displaying responses to PDGF signaling (FIG. 5E). Temporal lobe tumors samples, on the other hand, more commonly fell into the PDGF-‘non-responding’ group. In addition, examination of migration of GB cells from various samples revealed that higher alignment to laminin-coated patterns correlated with longer times until recurrence (FIG. 5F, FIG. 13C). This relationship was also confirmed using a univariate Cox analysis (P=0.002). As alignment is associated with the strength of cell—substrate adhesion (20, 30), this result could reflect a higher propensity in more aligned cells to adhere to ECM, leading to retarded migratory response and delayed tumor spread. A similar, yet weaker correlation was also observed between alignment and survival (P=0.09, univariate Cox analysis), however such relationships can be confounded by causes of death that are unrelated to cancer. Blind, qualitative analysis of patient tumor samples by a neuropathologist revealed small cell features, and marked microvascular proliferation in several samples of the PDGF-responding group. These higher order tumor structures are commonly associated with advanced progression, RTK amplification, and worse prognosis. On average, PDGF-responding samples indeed emerged from larger tumors that presented at younger ages and resulted in shorter survival and recurrence times (FIG. 6, FIG. 13A and 13B). Taken together, these findings highlight the translational potential of the proposed phenotypic analysis method as a screening tool for clinical evaluations, such as tissue biopsies.

Invasive nature of glioblastoma and other aggressive cancers highlights the importance of assaying cell migration as a phenotypic feature potentially predictive of clinical outcomes. Here we describe a simple but information-rich experimental method aimed at the analysis of primary patient samples on a single cell level, which allows high throughput screening of the effects of variable extracellular milieu. Using this method on a range of patient derived samples and contrasting the results of the analysis with respective clinical information revealed substantial predictive power, particularly when cell migration was examined in conjunction with the effects of PDGF. This result strongly suggests that cell migration, as examined in structured, mechanically-defined culture conditions, can indeed be predictive of more complex in vivo invasion processes and can thus be a powerful phenotypic analysis tool with strong clinical implications.

The experimental platform described in this report has important advantages over other phenotypic analysis platforms designed to assay cell invasion. For instance the trans-well migration analysis, another relatively simple method directly assaying cell invasion, for which a multi-well design has also been described, usually requires at least an order of magnitude greater numbers of cells than the method we describe here. More importantly, classical trans-well assays fail to yield the information on migration and morphology of each individual cell. This missing information can be critical in the analysis of human tumors. Indeed, we found a substantial degree of heterogeneity in the glioblastoma samples analyzed. The increased average migration speed of a given cell population in the presence of PDGF was ascribed to a relatively small sub-population of particularly aggressive cells (approximately 20%). Knowledge of the degree of population heterogeneity can be critical to the decision making in the clinic. Furthermore, we found that both speed and trajectory of individual cells within our platform were predictive of time to recurrence of tumors in the patients whose samples were analyzed. This information could be particularly useful in prognostic analyses of tumor samples at the time of surgery. Direct access to individual cell migration, available in the presented platform, would potentially gain critical importance.

The analysis presented here revealed the importance of careful engineering of extracellular milieu in cell migration analysis, both chemical and mechanical. Although sensitivity of cell movements to the presence of PDGF was a strong discriminating factor for various in vivo tumor characteristics, this sensitivity was more detectable when cells were cultured on adhesion substrata with fine, nano-scale topography designed to mimic the structure of in vivo extracellular cell micro-environment. This finding suggests that chemical and mechanical cues can strongly synergize both in vivo and in vitro to guide cell responses, necessitating flexible and multi-faceted bio-mimetic screening of multiple conditions in assaying patient samples. Patterning the nano-topographic features in the form of parallel nano-scale ridge arrays had an added advantage of simplifying the analysis of cell migration, as cells moved primarily in one-dimensional paths consistent with the orientation of this mechanical cue. This aspect of the experimental analysis makes the platform described here easy to use in both academic and clinical settings.

Thus far we have described a very precise design of the multi-well nanopatterned device. However, our novel design of this product includes a much larger range of features for which data has not been directly shown. As discussed above, experiments were conducted under a simplified proof-of-concept framework to demonstrate the capabilities of the system.

Our design has been successfully developed with a range of nanoridges sizes from 20 nm in diameter to 2 microns across. This also includes arrays of varying size, rigidity and spacing/density of the ridge features. Some of these substrates include a few distinct pattern structures, while others form gradual changes between numerous structures.

The experiments described above primarily employed a single material, poly(urethane acrylate) (PUA), to form the patterned substrates. However, other materials, e.g. polydimethylsiloxane, can be harnessed for the production of these nanopatterned surfaces. A single methodology was described above to produce the patterned surfaces used in the device. In addition to the described methodology, UV-assisted capillary force lithography (CFL), additional methods can be used in the production of these patterns, e.g. injection-molding, and standard soft lithography.

The bulk of this report has focused on the use of the multi-well, nanopatterned device to analyze the migratory behavior of human brain cancer cells. However, this platform can be utilized to monitor other cell behaviors such as proliferation, by observing cell divisions. In addition, this platform and variations on the nanotopographic feature size can be used and optimized to analyze behaviors of other cells types, including those of other species.

Glioblastoma (GB) samples were donated by patients at Johns Hopkins Medical Institutions. Human tissues were obtained and utilized with approval of the Institutional Review Board (IRB). Brain tumor samples GB 221, GB 253, GB 276, GB318, GB 499, GB 501, GB 544, GB 549, GB 567, GB 609, GB 612, GB 626, GB 630, and GB 854 were derived from primary intraoperative tissues of patients undergoing surgery for glioblastoma. Tissue donors received no treatment prior to surgery. All tissue samples were pathologically confirmed as glioblastoma. Primary GB cells were cultured either as adherent astrocytes or spheroids, i.e. neurospheres, as specified by experiment and as previously described. GB astrocytes were cultured in Dulbecco's Modified Eagle Medium: Nutrient Mixture F-12 containing 2 mM L-glutamine, with added 50 U mL-1 penicillin, 50 mg mL-1 streptomycin, and 10% fetal bovine serum (Invitrogen). Neurospheres were cultured in Dulbecco's Modified Eagle Medium: Nutrient Mixture F-12 containing 2 mM Lglutamine, with added 50 U mL-1 penicillin, 50 mg mL-1 streptomycin, supplemented with B27, 20 ng mL-1 endothelial growth factor (EGF), and 20 ng mL-1 fibroblast growth factor (FGF).

The topographic nanopatterned substratum, consisting of parallel ridges 350 nm wide, 500 nm high, spaced 1.5 μm apart, was fabricated onto glass coverslips as previously described, using UVassisted capillary molding techniques. Prior to application of the poly(urethane acrylate) (PUA) mold, glass substrates were cleaned with isopropyl alcohol, rinsed in distilled deionized water, and dried in a nitrogen stream. Afterwards, a thin layer (˜100 nm) of an adhesive agent (phosphoric acrylate: propylene glycol monomethyl ether acetate/41:10, volume ratio) was spin-coated onto the glass substrate for 30 s at 3000 rpm. Subsequently the PUA precursor was dispensed onto the substrate, and a previously constructed PUA mold was directly placed onto the surface. The PUA precursor spontaneously absorbed into the cavities of the mold via capillary action and was cured by exposure to UV light (λ=250-400 nm) for ˜30 s (dose=100 mJ cm-2).

To construct the multi-well device, nanopattern-coated glass coverslips were irreversibly bonded to modified Nunc® Lab-Tek® II Chamber Slide (cat. no. 154534) using biocompatible medical adhesive. Injection molding techniques can also be harnessed to fabricate multi-well chambers of various sizes and quantities for integration with the nanopattern-coated glass coverslips. Prior to attachment, pattern-coated glass coverslips were washed with 70% and 100% ethanol (EtOH), and allowed to air dry in a sterile environment. During and after construction, multi-well, nanopattern devices were maintained under sterile conditions.

Cells were cultured in the multi-well, nanopatterned device for approximately 48 hours in the course of each experiment. Prior to plating cells, nanoridged substrata were coated with poly-D-lysine (10 μg ml-1) for 15 minutes and mouse laminin (from 10 to 140 μg ml-1) for 1 hour. These topographically patterned cell substrata, caused cells to align with and move along the direction of the nanoridges. Both prior to, and during experiments cells were maintained at 37° C. and 5% CO2 in Dulbecco's Modified Eagle Medium: Nutrient Mixture F-12 containing 2 mM L-glutamine, with added 50 U mL-1 penicillin, 50 mg mL-1 streptomycin. Where indicated, media contained 10% fetal bovine serum (Invitrogen) or alternatively, Platelet-Derive Growth Factor-AA (PDGF-AA) (LC Laboratories) at specified concentrations.

Cell migration was observed using time-lapse microscopy (Movies S1-S3). To enable long-term observation, the multi-well, nanopatterned device was mounted onto the stage of a motorized inverted microscope (Olympus IX81) equipped with a Photometrics® Cascade® 512B II CCD camera and temperature and gas controlling environmental chamber. Phase contrast cell images were automatically recorded under 10× objective (NA=0.30) using the SlideBook™ 4.1 Software (Intelligent Imaging Innovations, Denver, Colo.) for 10-15 hours at 10 or 20 minute intervals.

Because cell-cell contact is known to affect the extent of cell spreading and migration, cells were plated at low density (˜4×104 cells mL -1) to allow isolated movements. A custom-made MATLAB® script was used allow manual tracking and measurement of cells frame by frame. Analysis was stopped in the event of cell death, division, collision with non-cellular debris, or movement out of the field of view. Area and spindle shape factor of individual cells was measured in each frame, and averaged over the entire duration of the experiment. The spindle shape factor was defined as the ratio of the maximum cell length (long axis) to the maximum cell width in the direction perpendicular to long axis, regardless of the orientation with respect to nanoridge pattern. Averages of cell populations were calculated from at least 60 cells.

Cells movements were tracked by centroid position or by the approximate center of the cell body. Individual cell trajectories were used to calculate the mean squared displacement (MSD) at each interval. Instantaneous speeds of individual cells were calculated from the MSD and the duration of the image acquisition time interval. Average speeds of individual cells were calculated from the total distanced moved throughout the entire cell trajectory and the total time the cell was tracked. Persistence was obtained as previously described, by calculating the ratio of the shortest distance between starting point and end point, divided by the total distanced moved. Alignment to the nanoridge pattern was calculated by dividing the distance moved parallel to the ridges, by the distance moved perpendicular to the ridges. Averages of cell populations were calculated from at least 60 cells.

Platelet-derived growth factor-AA ligand (PDGF-AA) was purchased from R&D Systems (10 ug) and reconstituted in 500 ul of 0.1% BSA. Imatinib, Methanesulfonate Salt was purchased from LC Laboratories. A 10 mM stock solution was dissolved in distilled water and stored at −20° C., protected from light. Dilutions of the stock for both PDGF and Imatinib were prepared for use in cell culture medium and added directly to the cells when needed.

Total RNA was isolated from cell lysates by homogenizing cells with 1 mL of Trizol (Invitrogen) and incubating in 200 μl of Chloroform. Cells were incubated overnight in isopropyl alcohol and RNA was extracted using the RNeasy® Mini Kit according to the manufacturer's instructions (Qiagen). A total of 1 μg of total RNA was reverse-transcribed into cDNA (Superscript III; Invitrogen). Polymerase chain reaction (PCR) amplifications were performed with a Platinum® Pfx DNA Polymerase (Invitrogen) in a PCR thermal cycler (Geneamp PCR System 9700, Applied Biosystems). After PCR amplification (5 minutes at 94° C. initial step, followed by 40 cycles of 15 seconds at 94° C., 40 seconds at 55° C., 30 seconds at 68 ° C. followed by 7 minutes at 68° C.), PCR products were analyzed on a 1.5% agarose gel (Invitrogen) containing SYBR® Safe DNA gel stain (Invitrogen) and imaged with Gel Logic 100 Imaging System (Kodak). Quantitative RT-PCR was performed using SYBR® Green PCR Master Mix (Applied Biosystems) and 7300 Real Time PCR Systems (Applied Biosystems). The thermal cycling conditions were as follows: 50° C. for 2 minutes, 95° C. for 10 minutes followed by 40 cycles of 95° C. for 15 seconds, 60° C. for 30 seconds, 72 ° C. for 30 seconds and finalized with 72° C. for 10 minutes. GAPDH was amplified as endogenous control. The sequence of PDGF Receptor-α primers employed is: sense, 5′-CCT GGT CTT AGG CTG TCT TCT-3′; antisense, 5′-GCC AGC TCA CTT CAC TCT CC-3′. The GAPDH primers' sequence is: sense, 5′-CAT GAG AAG TAT GAC AAC AGC CT-3′; antisense, 5′-AGT CCT TCC ACG ATA CCA AAG T-3′.

Neurosphere cells and astrocytes were grown on cover slips. The cells were fixed with 4% paraformaldehyde for 30 minutes at room temperature and permeabilized with PBS containing 0.1% Triton X-100 for 5 minutes. The cells were incubated overnight with primary antibodies for PDGF Receptor alpha (1:100; Santa Cruz) and then incubated with the appropriate secondary antibody conjugated with fluorescent dye (1:500) for one hour. Cells were subsequently stained against DAPI (1:200). Coverslips were mounted with Aquamount.

15-20 neurospheres were placed in DMEM/F12 without growth factors for 18 hours and then exposed to PDGF-AA ligand for 24 hours. Whole neurospheres were fixed with 4% paraformaldehyde for thirty minutes at room temperature and stained against Ki67 (1:200; Thermo) as previously described.

Total cellular protein was extracted using NE-PER Nuclear and Cytoplasmic Extraction Reagents kit according to the manufacturer's instructions (Thermo Scientific) containing protease (Roche) and phosphatase inhibitor (Thermo). Protein concentration was determined using the Bradford protein quantification method (Biorad Protein Assay, Biorad). SDS-PAGE was performed with 25 μg total cellular protein per lane using 4-12% gradient Tris glycine gels. The primary antibodies used were as follows: anti PDGFR-alpha (1:200; Santa Cruz); phospho-PDGFR alpha (1:1000; Cell Signaling); Akt (1:1000; Cell Signaling);

phospho-Akt (1:1000; Cell Signaling).

Edu incorporation was used as a measure of proliferation. For EdU incorporation experiments, cells (6×105) were cultured in DMEM/F12 medium without growth factors for 18 hours in a six well plate. Cells were exposed to EdU (10 μM Click-iT® EdU Flow Cytometry Assay Kit, Invitrogen) and PDGF-AA ligand (20 ng/mL) or a combination of PDGF-AA (20 ng/mL) and Imatinib (10 uM) for 24 hours. After incubation cells were centrifuged, the supernatant was discarded and the pellet suspended in 100 ul of 4% paraformaldehyde for 15 minutes. Cells were washed in 1% BSA and incubated with 100 ul of saponinbased permeabilization buffer for 10 minutes. After an additional wash in 1% BSA cells were incubated in 250 ul of Click-iT® Reaction Cocktail for 30 minutes. All procedures were performed according to manufacturer's instructions except for the volumes used to prepare the Click-iT® Reaction buffer. Half of the recommended volume was used for all reagents.

Flow cytometry was performed using a FACSCaliber™ Flow Cytometer (BD Biosciences) and data was analyzed with Kaluza® Flow Cytometry Software (Beckman Coulter). Analysis of 30,000 total events was performed after exclusion of dead cells by FSC/SSC gating. Fluorescence was measured in the FL4 channel.

Cell invasion was evaluated by means of transwell migration assays in Growth Factor Reduced BD Matrigel Invasion Chambers (BD Biochemicals). Cells were dissociated and 100,000 cells/mL were resuspended in DMEM plus 0.5% serum media with or without PDGF-AA (20 ng/mL). Cell suspension aliquots (500 μl) were plated onto the upper filter surface of the transwell plates with a porous PET membrane (8 μm pores) coated with a layer of growth factor reduced matrigel basement membrane matrix. 750 μl of DMEM plus 2% serum was placed on the lower chamber. Filters were incubated at 37C° , 5% CO2/95% air condition for 48 hours. Migration of cells was determined by fixing the membrane, staining the cells using the Diff Quik® staining kit, directly counting the number of migration cells in 13 high-power fields at 10× using an Olympus 1X81 microscope system and calculating the mean. The assays were run in triplicates and three independent experiments were performed.

All animal protocols were approved by the Johns Hopkins School of Medicine Animal Care and Use Committee. For intracranial xenografts, SCID immnodeficient mice received 100,000 viable cells in 1 μl of DMEM/F12 serum media without growth factors by stereotactic injection into the right striatum. Cells were cultured in DMEM/F12 serum media with EGF/FGF and PDGF-AA ligand for three weeks before injections were performed. Cell viability was determined by trypan blue dye exclusion. Mice were perfused with 4% paraformaldehyde at the indicated times and the brains were removed for histological analysis.

Results are presented as mean+s.e.m. Mann-Whitney Rank Sum Test was for pairwise comparisons; Dunn's Test (rank-based ANOVA) was used in multiple group comparisons. In some cases, as noted, Student's t-test or standard ANOVA, Holm-Sadik Method was used. Univariate Cox analysis was used to identify correlations between tumor characteristics. To group certain data, thresholds were determined using linear discriminant analysis as previously described. Statistics were analyzed using Sigmaplot®, GraphPad Prism®, and MATLAB® software.

The technology currently uses an 8-well construction to allow analysis of multiple samples simultaneously. We plan to increase this number to as high as 96 wells, to increase the throughput of the assay. The technology currently uses a single pattern design. These are parallel nano-ridges with rectangular cross-section and the follow dimensions: 350 nm wide, 500 nm high, spaced 1.5 μm apart. We plan to include pattern designs of various sizes including: 400 nm wide, 400 nm high, spaced 400 nm apart.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for cell migration comprising:
a multi-well plate having a number of well chambers, wherein the well chambers comprise at least one wall defining the well chamber;
a nanopatterned surface, wherein the nanopatterned surface is positioned on the at least one wall defining the well chamber;
wherein the nanopatterened surface is configured to mimic the topographic structure of a natural cell environment.

2. The device for cell migration of claim 1 wherein the nanopatterned surface further comprises parallel ridges.

3. The device for cell migration of claim 2 wherein the parallel ridges are approximately 350 nm wide and approximately 500 nm high and spaced apart 1.5 μm.

4. The device for cell migration of claim 1 wherein the nanopatterned surface is formed directly into the at least one wall defining the well chamber.

5. The device for cell migration of claim 1 wherein the nanopatterened surface is formed onto a glass coverslip.

6. The device for cell migration of claim 1 wherein the nanopatterned surface is configured to mimic the mechanical signals of natural ECM topography.

7. The device for cell migration of claim 6 wherein the nanopatterned surface configured to mimic the mechanical signals of natural ECM topography is combined with chemical cues.

8. The device for cell migration of claim 7 wherein the chemical cues are soluble.

9. The device for cell migration of claim 7 wherein the chemical cues are immobilized.

10. The device for cell migration of claim 1 further comprising a coating of laminin.

11. The device for cell migration of claim 10 further comprising varying the concentration of laminin in the coating.

12. The device for cell migration of claim 1 further comprising a coating of platelet derived growth factor (PDGF).

13. The device for cell migration of claim 12 further comprising varying the concentration of PDGF in the coating.

14. The device for cell migration of claim 1 further comprising configuring the device for cell migration to test migration of glioblastoma.

15. The device for cell migration of claim 1 further comprising configuring the device to provide patient-specific results regarding tumor progression.

16. The device for cell migration further comprising configuring the device for diagnosis of brain tumors.
